(19) United States
(12) Patent Application Publication
(10) Pub. No.: US 2008/0221434 A1
(43) Pub. Date: Sep. 11, 2008

(54) Displaying an internal image of a body lumen of a patient

(76) Inventor: James W. Voegele, Cincinnati, OH (US)

Correspondence Address: THOMPSON HINE L.L.P.;Intellectual Property Group, P.O. BOX 8801, DAYTON, OH, 45401-8801, US

(21) Appl. No.: 11/716,465

(22) Filed: Mar. 9, 2007

(51) Int. Cl. A61B 5/05

(52) U.S. Cl. 600/424

(57) ABSTRACT

A medical system includes a display monitor, a catheter, a sensor, non-light-obtained image data of a patient, and a computer. The catheter has a distal end insertable into a body lumen of the patient. The sensor is attached to the catheter and is adapted to provide position data. The computer is adapted to calculate a position of the sensor using at least the position data indexed to a reference coordinate system, to create an internal image representation of the body lumen using at least the image data indexed to the reference coordinate system, and to display on the monitor a display image of the internal image representation of the body lumen. A storage medium contains a program readable by a computer which instructs the computer to perform the previously described steps. A method for visualizing a position of a catheter within a patient performs the above-described steps.

### FIELD OF THE INVENTION

The present invention is related generally to medical images, and more particularly to a medical system, to a storage medium containing a computer program, and to a method all relating to displaying an internal image of a body lumen of a patient.

### BACKGROUND OF THE INVENTION

A physician typically accesses and visualizes tissue within a patient's gastrointestinal (GI) tract with an endoscope (such as a gastroscope or a colonoscope) having a long, flexible insertion tube. For the upper GI, a physician may insert a gastroscope into the sedated patient's mouth to examine and treat tissue in the esophagus, stomach, and proximal duodenum. For the lower GI, a physician may insert a colonoscope through the sedated patient's anus to examine the rectum and colon. The light-obtained images from a video camera at the distal end of the insertion tube are displayed on a monitor for use by the physician. Some endoscopes have a working channel in the insertion tube extending from a port in the handpiece to the distal portion of the insertion tube. A physician may insert medical devices into the working channel to help diagnose or treat tissue within the patient. Non-endoscope type catheters are known which do not have a video camera and which either have a working channel for insertion of a medical device therein or have an attached end effector defining the distal end of the catheter.

Imagers are known for obtaining image data of a patient and for displaying images of the image data on a display monitor. Such images include, without limitation, ultrasound images, X-ray images, computerized tomography (CT) images, positive electron emission (PET) images, magnetic resonance (MRI) images, fluoroscope images, etc. Where needed, it is known to register these images with a real world object by placing a marker on the skin of the patient, wherein the marker has a predetermined shape, and wherein the marker is recognizable in the image data using pattern recognition software (e.g., a conventional segmentation subroutine).

Position sensors are known which are placed on medical instruments which are inserted into a patient allowing the position of the medical instrument to be tracked inside the patient. Such position sensors are part of known position sensing systems such as an AC-based system available from Biosense-Webster or a DC-based system available from Ascension Technology Corporation.

Still, scientists and engineers continue to seek improved medical systems, computer programs, and methods for displaying medical images.

### SUMMARY

A first expression of an embodiment of a medical system of the invention is for a medical system which includes a display monitor, a catheter, a sensor, non-light-obtained image data of a patient, and a computer. The catheter has a distal end insertable into a body lumen of the patient. The sensor is attached to the catheter and is adapted to provide position data. The computer is adapted to calculate a position of the sensor using at least the position data indexed to a reference coordinate system, to create an internal image representation of the body lumen using at least the image data indexed to the reference coordinate system, and to display on the display monitor a display image of the internal image representation of the body lumen.

A first expression of an embodiment of a storage medium of the invention is for a storage medium which contains a program readable by a digital computer which instructs the digital computer to: calculate a position of a sensor using at least position data obtained from the sensor and indexed to a reference coordinate system, wherein the sensor is attached to a catheter having a distal end insertable into a body lumen of a patient; create an internal image representation of the body lumen using at least non-light-obtained image data obtained from the patient and indexed to the reference coordinate system; and display on a display monitor a display image of the internal image representation of the body lumen.

A first expression of a method of the invention is for a method for visualizing a body lumen of a patient when a distal end of a catheter is disposed in the body lumen and includes: calculating a position of a sensor using at least position data obtained from the sensor and indexed to a reference coordinate system, wherein the sensor is attached to the catheter; creating an internal image representation of the body lumen using at least non-light-obtained image data obtained from the patient and indexed to the reference coordinate system; and displaying on a display monitor a display image of the internal image representation of the body lumen.

Several benefits and advantages are obtained from one or more expressions of the embodiment of the system, the embodiment of the storage medium, and the method of the invention. In one example, the image data is pre-acquired image data and the position data is real-time position data. In the same or a different example, the internal image representation of the body lumen faces substantially along the centerline of the body lumen, the centerline of the body lumen of the display image is kept substantially centered on the display monitor, and the display image changes with changes in insertion position of the catheter in the body lumen providing a “tunnel-like” moving image of the body lumen seen from inside the body lumen from the “viewpoint” of the distal end of the catheter. In one utilization, the displayed image allows the physician to better guide the catheter (especially a catheter not having a video camera or fiber optics) in the body lumen and to identify treatment sites in the body lumen.

### BRIEF DESCRIPTION OF THE FIGURES

FIG. 1 is a schematic view of an embodiment of a medical system of the invention, wherein a sensor is attached to the catheter of the medical system proximate the distal end of the catheter;

FIG. 2 is an example of a display image of an internal image representation of a body lumen of a patient (which is a display image seen from inside the body lumen) which is displayed on the display monitor by one enablement of a method of the invention using the medical system of FIG. 1, wherein arrows indicate the display image is a “tunnel-like” moving image of the body lumen from within the body lumen from the “viewpoint” of the distal end of the moving catheter; and

FIG. 3 is a block diagram of a method of invention which, in one example, is incorporated into a program contained in a storage medium of the digital computer of the medical system of FIG. 1.

### DETAILED DESCRIPTION

Before explaining the system embodiment, the computer program steps, and the method of the present invention in detail, it should be noted that each is not limited in its application or use to the details of construction and arrangement of parts and steps illustrated in the accompanying drawings and description. The illustrative system embodiment, computer program steps, and method of the invention may be implemented or incorporated in other embodiments, computer programs, methods, variations and modifications, and may be practiced or carried out in various ways. Furthermore, unless otherwise indicated, the terms and expressions employed herein have been chosen for the purpose of describing the illustrative embodiments and method of the present invention for the convenience of the reader and are not for the purpose of limiting the invention.

It is further understood that any one or more of the following-described system embodiment, computer program steps, method, implementations, etc. can be combined with any one or more of the other following-described system embodiment, computer program steps, method, implementations, etc.

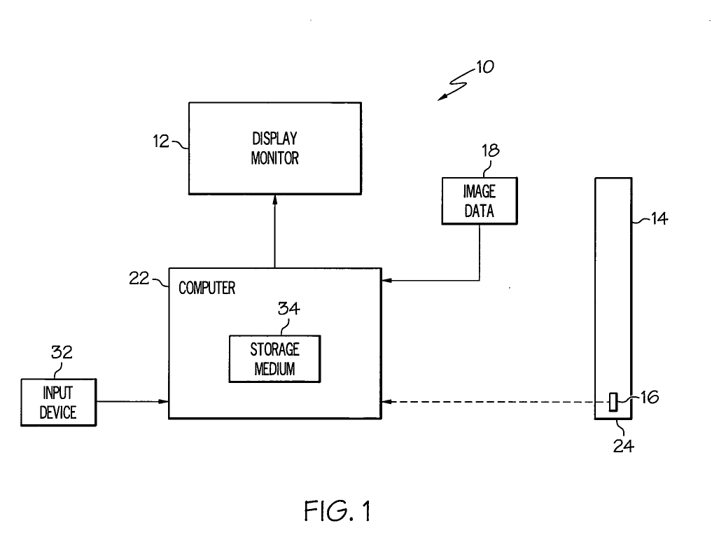

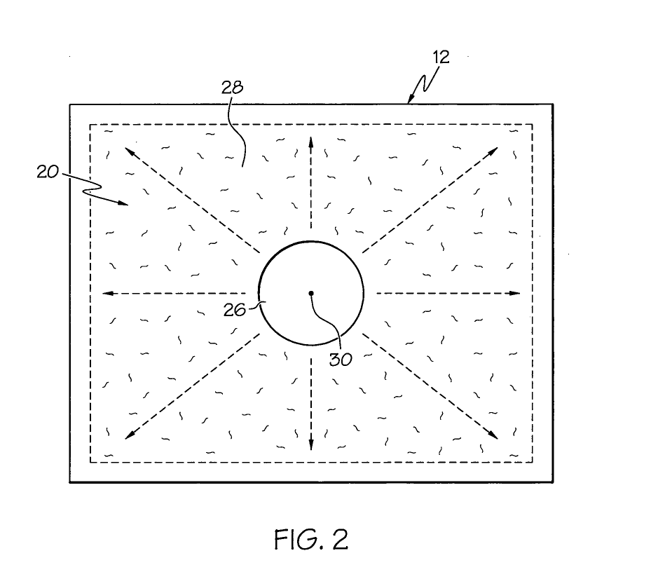

An embodiment of a medical system 10 of the invention is shown in FIGS. 1-2. A first expression of the system embodiment of FIGS. 1-2 is for a medical system 10 including a display monitor 12, a catheter 14, a sensor 16, non-light-obtained image data 18 of a patient 20, and a computer 22. The catheter 14 has a distal end 24 insertable (i.e., capable of being inserted) into a body lumen 26 of the patient 20. The sensor 16 is attached to the catheter 14 and is adapted to provide position data. The computer 22 is adapted to calculate a position of the sensor 16 using at least the position data indexed to a reference coordinate system, to create an internal image representation of the body lumen 26 using at least the image data 18 indexed to the reference coordinate system, and to display on the display monitor 12 a display image 28 of the internal image representation of the body lumen 26.

In one realization of the first expression of the system embodiment of FIGS. 1-2, the position data and/or the image data 18 are already indexed to the reference coordinate system when received by the computer 22. In a different realization, the position data and/or the image data 18 are not yet indexed to the reference coordinate system when received by the computer 22, and such indexing is performed by the computer 22.

Examples of non-light-obtained image data 18 include, without limitation, ultrasound images, X-ray images, computerized tomography (CT) images, positive electron emission (PET) images, magnetic resonance (MRI) images, and fluoroscope images. An example of a computer program which creates a manipulative 3D display image from 2D CT-scans and MRI-scans is Mimics available from Materialise of Ann Arbor, Mich. Examples of a display monitor 12 include, without limitation, a computer monitor, a goggle display screen, and a room wall upon which projected images are displayed.

Examples of catheters 14 include, without limitation, cardio-vascular catheters, pulmonary catheters, and flexible insertion tubes of endoscopes such as insertion tubes of gastroscopes and colonoscopes. In one variation, the catheter 14 is equipped with a centering means, such as a balloon, so the catheter 14 will travel down the center of the body lumen 26. It is noted that, as used in describing the system embodiment of FIGS. 1-2, the terminology “body lumen” is any hollow internal structure of the patient 20. Examples of a body lumen 26 of a patient 20 include, without limitation, the upper GI (gastrointestinal) tract, the lower GI tract, a lung, a urinary tract, and a blood vessel passageway. Other examples of catheters 14 and/or body lumens 26 are left to the artisan.

Examples of sensors 16 adapted to provide position data include, without limitation, the position sensors of the AC-based position sensing system available from Biosense-Webster and the DC-based position sensing system available from Ascension Technology Corporation. It is noted that, as used in describing the system embodiment of FIGS. 1-2, the term “position” includes up to six degrees of freedom so that calculating position includes calculating a two-dimensional or three-dimensional translation and two or three degrees of orientation of the sensor 16 with respect to a reference coordinate system. A description of the operation of an embodiment of a sensor 16 adapted to provide position data is found in US Patent Application Publication 2006/0089624.

In one illustration of the first expression of the system embodiment of FIGS. 1-2, the sensor 16 is considered to be a position sensor of a Biosense Webster positioning sensing system and a transmitter, not shown, of such system is used by the computer 22 for a reference coordinate system for position data from the sensor 16. Thus, the computer 22 can index the position data of the sensor 16 to the reference coordinate system.

In this illustration, a marker-sensor assembly, not shown, is placed on the patient 20, wherein the marker portion shows up on the image data 18 of the patient 20, is identifiable by a conventional segmentation subroutine running on the computer 22, and serves to relate the image data 18 to the real world marker. In one example, the sensor portion of the marker-sensor assembly is another position sensor of the Biosense Webster positioning sensing system and provides position data of the marker-sensor assembly to the computer 22. Therefore, the image data 18 is related to (the marker portion of) the marker-sensor assembly and the position of (the sensor portion of) the marker-sensor assembly is related to the reference coordinate system. Thus, the computer 22 can index the image data 18 to the reference coordinate system. As the position data of the sensor 16 and the image data 18 of the patient 20 are both indexed to the same reference coordinate system, a subroutine can be written by those of ordinary skill in the art, without undue experimentation, which instructs the computer 22 to display a display image 28 of the internal image representation of the body lumen 26.

In a first variation of this illustration, the display image 28 is an image of the internal image representation of the body lumen 26 as seen from the viewpoint of the sensor 16 (i.e., the image is registered with [superimposed on] the [non-displayed or displayed] position of the sensor 16). In a second variation, the display image 28 is an image of the internal image representation of the body lumen 26 as seen from the viewpoint of the center of the distal end 24 of the catheter 14 (i.e., the image is registered with [superimposed on] the [non-displayed or displayed] center of the distal end 24 of the catheter 14). Other variations are left to the artisan.

In one enablement of the first expression of the system embodiment of FIGS. 1-2, the image data 18 is pre-acquired image data, and the position data is real-time position data.

In the same or a different enablement, the sensor 16 is the only sensor of the medical system 10 which is attached to the catheter 14 and adapted to provide position data. In one variation, the sensor 16 is attached to the catheter 14 proximate the distal end 24 of the catheter 14. In one modification, the sensor 16 is attached to the catheter 14 distal of any articulation joint of the catheter 14, wherein the catheter 14 from the sensor 16 to the distal end 24 is rigid.

In the same or a different enablement, the body lumen 26 has a centerline 30 (which has been added for clarification to FIG. 2 appearing as a dot because FIG. 2 is a view seen looking along the centerline), and the internal image representation of the body lumen 26 created by the computer 22 faces substantially along the centerline 30 of the body lumen 26. In one variation, the computer 22 is adapted to substantially center (but not show) the centerline 30 on the display monitor 12 (which will prevent the image from jumping around since breathing and other anatomical motion would be negated). In the same or a different variation, such internal image representation faces distal the distal end 24 of the catheter 14. In one modification, the display image 28 changes with changes in insertion position of the catheter 14 in the body lumen 26. In this modification, the display monitor 12 shows a “tunnel-like” moving display image 28 of the body lumen 26 seen from inside the body lumen from the “viewpoint” of the moving catheter 14. The un-numbered dashed arrowhead lines in FIG. 2 indicate such “tunnel-like” movement which is similar to the “tunnel-like” movement seen in “starfield” computer screen savers.

In one utilization of the first expression of the embodiment of FIGS. 1-2, the sensor 16 provides the position data, and the computer 22 calculates the position of the sensor 16, creates the internal image representation of the body lumen 26, and displays on the display monitor 12 the display image 28. In a first variation, the time frequency, for the computer 22 to update the display image 28 displayed on the display monitor 12, is a user input to the computer 22. In a second variation, the time frequency is a fixed number. In a third variation, the time frequency is determined by the computer 22 based on variables such as, but not limited to, the speed of the catheter 14.

In one application of the first expression of the embodiment of FIGS. 1-2, the computer 22 is adapted to create an internal image representation of the body lumen 26 with a translucency of the tissue of the body lumen 26 in order to also show internal body structure (such as one or more internal body organs) on the display image 28 which is beyond (i.e., outside) the wall of the body lumen 26. In one variation, the catheter 14 is an articulatable catheter whose distal end can be made to point to the side of the body lumen 26 to substantially directly face the wall of the body lumen 26, and the computer 22 is adapted to create the internal image representation of the body lumen 20 to face in the direction along which the distal end of the catheter is pointed, wherein internal body structure on the other side of the body lumen 26 is seen substantially “head-on” through the translucent-displayed wall of the body lumen 26 in the display image 28.

In an employment of the first expression of the embodiment of FIGS. 1-2, the display image 28 is a three-dimensional manipulative image, and the medical system 10 also includes a computer input device 32 operatively connected to the computer 22 to allow a user to manipulate the three-dimensional-manipulative image on the display monitor 12 (such as changing from a forward-looking view to a side-looking view inside the body lumen 26). Examples of input devices 32 include, without limitation, a keyboard and a mouse. In a different employment, the display image 28 is a two-dimensional non-manipulative image.

In one extension of the first expression of the embodiment of FIGS. 1-2, the computer 22 is adapted to calculate and to display (and in one utilization calculates and displays) on the display monitor 12 at least one numerical relationship derived from the position data and the image data 18. In one example, the at least one numerical relationship includes a countdown distance remaining between the distal end 24 of the catheter 14 and a particular point along the body lumen 26, such as the end of the esophagus. In a first variation, the particular point is identified to the computer 22 by a user moving a cursor over a displayed patient image created by the computer 22 from the image data 18 to include an area of interest, such as the esophagus, and clicking on a displayed point of interest, such as the end of the esophagus. In a second variation, the computer 22, using pattern-recognition software, identifies the particular point, such as the end of the esophagus, when (but not limited to) a user has touched “end of esophagus” from a list displayed on a touch screen portion of the display monitor 12. Other examples of numerical relationships include dimensions associated with the arcuate path of a body lumen 26, catheter 14 inserted length, and point-to-point and angular relationships of any relative features such as mouth to distal tip of catheter 14. Additional examples are left to the artisan.

A first expression of an embodiment of a storage medium 34 of the invention is for a storage medium 34 which contains a program readable by a digital computer 22 which instructs the digital computer 22 to perform steps a) through c). Step a) includes calculating a position of a sensor 16 using at least position data obtained from the sensor 16 and indexed to a reference coordinate system, wherein the sensor 16 is attached to a catheter 14 having a distal end 24 insertable into a body lumen 26 of a patient 20. Step b) includes creating an internal image representation of the body lumen 26 using at least non-light-obtained image data 18 obtained from the patient and indexed to the reference coordinate system. Step c) includes displaying on a display monitor 12 a display image 28 of the internal image representation of the body lumen 26.

It is noted that the enablements, applications, etc. of the previously-described first expression of the embodiment of the medical system 10 are equally applicable to the first expression of the embodiment of the storage medium 34. Examples of storage media include, without limitation, temporary computer memory and permanent computer memory such as RAM, hard drives, CD's, etc.

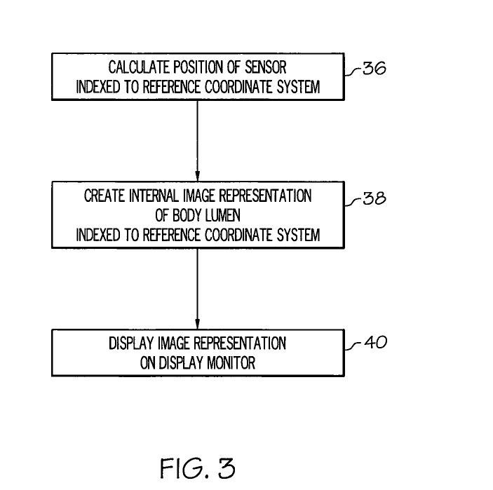

A method of the invention is for visualizing a body lumen 26 of a patient 20 when a distal end 24 of a catheter 14 is disposed in the body lumen 26. A first expression of the method is shown in FIG. 3 and includes steps a) through c) which are identical to the previously-described steps a) through c) of paragraph [0035]. Step a) is labeled as “Calculate Position Of Sensor Indexed To Reference Coordinate System” in block 36 of FIG. 3. Step b) is labeled as “Create Internal Image Representation Of Body Lumen Indexed To Reference Coordinate System” in block 38 of FIG. 3. Step c) is labeled as “Display Image Representation On Display Monitor” in block 40 of FIG. 3.

It is noted that the enablements, applications, etc. of the previously-described first expression of the embodiment of the medical system 10 are equally applicable to the first expression of the method.

Several benefits and advantages are obtained from one or more expressions of the embodiment of the system, the embodiment of the storage medium, and the method of the invention. In one example, the image data is pre-acquired image data and the position data is real-time position data. In the same or a different example, the internal image representation of the body lumen faces substantially along the centerline of the body lumen, the centerline of the body lumen of the display image is kept substantially centered on the display monitor, and the display image changes with changes in insertion position of the catheter in the body lumen providing a “tunnel-like” moving image of the body lumen seen from inside the body lumen from the “viewpoint” of the distal end of the catheter. In one utilization, the displayed image allows the physician to better articulate and guide the catheter (especially a catheter not having a video camera or fiber optics) in the body lumen and to identify treatment sites in the body lumen.

While the present invention has been illustrated by expressions of a system embodiment, a storage medium embodiment containing a program readable by a digital computer, and a method, and enablements, applications, etc. thereof, it is not the intention of the applicant to restrict or limit the spirit and scope of the appended claims to such detail. Numerous other variations, changes, and substitutions will occur to those skilled in the art without departing from the scope of the invention. It will be understood that the foregoing description is provided by way of example, and that other modifications may occur to those skilled in the art without departing from the scope and spirit of the appended Claims.

What is claimed is:

1. A medical system comprising:
a) a display monitor;
b) a catheter having a distal end insertable into a body lumen of a patient;
c) a sensor attached to the catheter and adapted to provide position data;
d) non-light-obtained image data of the patient; and
e) a computer adapted to calculate a position of the sensor using at least the position data indexed to a reference coordinate system, to create an internal image representation of the body lumen using at least the image data indexed to the reference coordinate system, and to display on the display monitor a display image of the internal image representation of the body lumen.

2. The medical system of claim 1, wherein the image data is pre-acquired image data and wherein the position data is real-time position data.

3. The medical system of claim 1, wherein the sensor is the only sensor of the medical system which is attached to the catheter and adapted to provide position data.

4. The medical system of claim 3, wherein the sensor is attached to the catheter proximate the distal end of the catheter.

5. The medical system of claim 1, wherein the body lumen has a centerline and wherein the internal image representation of the body lumen created by the computer faces substantially along the centerline of the body lumen.

6. The medical system of claim 5, wherein the computer is adapted to substantially center the centerline of the body lumen of the display image on the display monitor.

7. The medical system of claim 1, wherein the display image changes with changes in insertion position of the catheter in the body lumen.

8. A storage medium containing a program readable by a digital computer which instructs the digital computer to:
a) calculate a position of a sensor using at least position data obtained from the sensor and indexed to a reference coordinate system, wherein the sensor is attached to a catheter having a distal end insertable into a body lumen of a patient;
b) create an internal image representation of the body lumen using at least non-light-obtained image data obtained from the patient and indexed to the reference coordinate system; and
c) display on a display monitor a display image of the internal image representation of the body lumen.

9. The storage medium of claim 8, wherein the image data is pre-acquired image data and wherein the position data is real-time position data.

10. The storage medium of claim 8 wherein the sensor is the only sensor of the medical system which is attached to the catheter and adapted to provide position data.

11. The medical system of claim 10, wherein the sensor is attached to the catheter proximate the distal end of the catheter.

12. The storage medium of claim 8, wherein the body lumen has a centerline and wherein the internal image representation of the body lumen created by the computer faces substantially along the centerline of the body lumen.

13. The storage medium of claim 12, wherein the computer is adapted to substantially center the centerline of the body lumen of the display image on the display monitor.

14. The storage medium of claim 8, wherein the display image changes with changes in insertion position of the catheter in the body lumen.

15. A method for visualizing a body lumen of a patient when a distal end of a catheter is disposed in the body lumen comprising:
a) calculating a position of a sensor using at least position data obtained from the sensor and indexed to a reference coordinate system, wherein the sensor is attached to-the catheter;
b) creating an internal image representation of the body lumen using at least non-light-obtained image data obtained from the patient and indexed to the reference coordinate system; and
c) displaying on a display monitor a display image of the internal image representation of the body lumen.

16. The method of claim 15, wherein the image data is pre-acquired image data and wherein the position data is real-time position data.

17. The method of claim 15 wherein the sensor is the only sensor of the medical system which is attached to the catheter and adapted to provide position data and wherein the sensor is attached to the catheter proximate the distal end of the catheter.

18. The method of claim 15, wherein the body lumen has a centerline and wherein the internal image representation of the body lumen created by the computer faces substantially along the centerline of the body lumen.

19. The method of claim 18, wherein the computer is adapted to substantially center the centerline of the body lumen of the display image on the display monitor.

20. The method of claim 15, wherein the display image changes with changes in insertion position of the catheter in the body lumen.
